SEQUENCE LISTING (12 sequences)

SEQ ID NO: 1 - protein, 13 aa, Homo sapiens
  misc_feature (6)..(6): X / Xaa denotes citrulline
GGYRAXPAKAAAT

SEQ ID NO: 2 - protein, 13 aa, Artificial Sequence
  Synthetic peptide
GGYRAQPAKAAAT

SEQ ID NO: 3 - protein, 13 aa, Artificial Sequence
  Synthetic peptide
  misc_feature (6)..(6): X / Xaa denotes citrulline
GGFRAXPAKAAAT

SEQ ID NO: 4 - protein, 13 aa, Artificial Sequence
  Synthetic peptide
GGFRAQPAKAAAT

SEQ ID NO: 5 - protein, 13 aa, Homo sapiens
  misc_feature (6)..(6): X / Xaa denotes citrulline
SAVRLXSSVPGVR

SEQ ID NO: 6 - protein, 13 aa, Artificial Sequence
  Synthetic peptide
SAVRLQSSVPGVR

SEQ ID NO: 7 - protein, 13 aa, Artificial Sequence
  Synthetic peptide
  misc_feature (6)..(6): X / Xaa denotes citrulline
SAFRLXSSVPGVR

SEQ ID NO: 8 - protein, 13 aa, Artificial Sequence
  Synthetic peptide
SAFRLQSSVPGVR

SEQ ID NO: 9 - protein, 15 aa, Homo sapiens
  misc_feature (7)..(7): X / Xaa denotes citrulline
VSLISRXGDMSSNPA

SEQ ID NO: 10 - protein, 15 aa, Homo sapiens
  misc_feature (7)..(7): X / Xaa denotes citrulline
QGQYELXVDLRDHGE

SEQ ID NO: 11 - protein, 15 aa, Artificial Sequence
  Synthetic peptide
VSLISRQGDMSSNPA

SEQ ID NO: 12 - protein, 15 aa, Artificial Sequence
  Synthetic peptide
QGQYELQVDLRDHGE

(19) United States
(12) Patent Application Publication
(10) Pub. No.: US 2025/0340616 A1
(43) Pub. Date: Nov. 6, 2025

(54) NOVEL PEPTIDE MIMICS AND THEIR USE

(71) Applicant: STIFTELSEN VECTIS, Stockholm (SE)

(72) Inventors: Lars Klareskog, Stockholm (SE); Vivianne Malmström, Stockholm (SE); Anatoly Dubnovitsky, Uppsala (SE)

(21) Appl. No.: 18/565,905

(22) PCT Filed: Jun. 1, 2022
(86) PCT No.: PCT/SE2022/050527

§ 371 Date: Nov. 30, 2023

(30) Foreign Application Priority Data
Jun. 1, 2021 (SE) 2150702-5

(51) Int. Cl. C07K 14/75; A61K 39/00; C07K 14/47; C07K 14/78

(52) U.S. Cl. CPC C07K 14/75; A61K 39/0008; C07K 14/47; C07K 14/78; A61K 2039/53

(57) ABSTRACT

The present invention relates to mimics of post-translationally modified naturally occurring peptides, wherein said peptide mimics bind to a peptide-binding groove of human leukocyte antigen (HLA) molecules to the same extent as the naturally occurring post-translationally modified peptide, wherein said peptide mimics are recognized by T cells to the same extent as the naturally occurring post-translationally modified peptide, and additionally wherein said peptide mimics have a three-dimensional structure substantially identical to said post-translationally modified naturally occurring peptide. Such peptide mimics can be used alone of bound to a carrier, and have utility inter alia in methods for the treatment, alleviation and prevention of autoimmune diseases, and as components in tolerogenic vaccines.

### TECHNICAL FIELD

This disclosure relates to the field of medicine, more specifically immunotherapy and in particular novel synthetic mimics of post-translationally modified naturally occurring peptides, MHC class II-peptide complexes including vaccines and other compositions comprising such mimics, and their use for example in therapeutic and prophylactic methods for induction of tolerance against a specific antigen in a subject.

### BACKGROUND

Much effort has been invested in the development of immunotherapy for the treatment and prevention of autoimmune diseases. The ultimate goal would be the induction of antigen-specific tolerance. Increasing awareness and accumulating knowledge of the antigens that are recognized by potentially pathogenic T and B cells has recently made this goal appear more attainable. Ideally, such knowledge could make it possible to develop antigen-specific therapies that eliminate or re-regulate such pathogenic immunity.

Of particular interest in this development is detailed knowledge of the peptides that bind to specific allelic forms of major histocompatibility complex (MHC) class II molecules where these peptides are recognized by potentially pathogenic T cells from patients. One procedure for tolerization that is currently being developed by several academic and pharma/biotech groups is based on preparing such constructs that contain MHC class II-peptide complexes. These complexes can then be administered to the patient together with suitable carriers to induce antigen-specific tolerance.

In 2012, WO2012138294A1 presented novel peptides from human alpha-enolase, collagen type II and vimentin capable of binding to different types of MHC class II molecules.

Published in 2013, the application AU2013204094A1 titled “Citrullinated peptides for diagnosing and prognosing rheumatoid arthritis” presented a mimic of a post-translationally modified naturally occurring 9-residue peptide within the vimentin polypeptide, wherein an arginine residue was replaced with glutamine to mimic citrullination.

Harauz G. and Musse A. A. et al., 2006 investigated the post-translational modifications of myelin basic protein (MBP) and found inter alia that the degree of deamination (or citrullination) of MBP is correlated with the severity of MS. There is no information concerning possible binding to HLA or recognition by T-cells.

A review of emerging treatments is given by Nel et al., in Lancet Rheumatology, 2020. In this article, the authors discuss the results of early-stage clinical trials indicating that immunotherapy might allow extended duration of remission and even prevention of progression to disease, suggesting that modulating tolerance in rheumatoid arthritis could be a promising opportunity for therapy.

### SUMMARY

While the above approach may be promising for many autoimmune diseases, the present inventors have realized that the only peptides so far described as relevant for rheumatoid arthritis (RA) and binding to the appropriate MHC molecules are post-translationally modified, i.e. a citrulline is necessary as an amino acid involved either in the binding to the MHC molecule or involved in the recognition of the peptide-MHC complex by an RA-derived T-cell receptor (TCR).

A major problem in producing such therapeutic MHC class II peptide complexes, or other products that require the synthesis of post-translationally modified amino acids, has been that the procedure for making these products requires a step of post-translational modification after the initial synthesis. One example of this is the synthesis of the combined MHC molecule and the peptide binding to the peptide-binding groove of this molecule. Production of such complexes is not possible if a critical amino acid in this peptide is post-translationally modified, and this feature is thus a major hurdle for the development of potential therapeutic MHC class II peptide containing constructs.

The same problem, but in another technical context, relates to the potential to use mRNA based vaccines for tolerization. As shown in a recent article from BioNTech (Krienke et al., 2021) an mRNA vaccine coding for peptides relevant for tolerization in the Experimental Allergic Encephalomyelitis model, such mRNA vaccines (without the tag that activates the immune system as is used for example in COVID vaccines) can also induce an antigen-specific tolerance. In this case, it is not possible to make a mRNA vaccine for the treatment of RA as it is not possible to produce post-translationally modified amino acids from mRNA codes.

Based on their knowledge of the crystal structure of certain relevant allelic variants of MHC class II (HLA-DR0401 and DR0404), relevant citrullinated peptides and where these citrullinated peptides are recognized by T cells from RA patients, the present inventors however succeeded in synthesizing novel, synthetic and alternative non-citrullinated peptide mimics that both bind to the peptide-binding groove of HLA-DR0401 and 0404 and are recognized by T cell receptors derived from T cells that are activated in RA patients.

The inventors also developed systems for the identification of peptides that bind to RA-relevant allelic forms of MHC class II molecules (HLA-DR) and used T cell clones generated from RA patients and recognizing the appropriate MHC class II-citrullinated peptide complex, for testing of whether the novel peptidomimetics (a peptide not containing citrulline) were able to both bind to the appropriate MHC class II molecule and be recognized by T cell clones from RA patients.

Accordingly, a first aspect of the present disclosure concerns a synthetic mimic of a post-translationally modified naturally occurring peptide, wherein a citrulline has been substituted with another amino acid forming a peptide mimic, wherein said peptide mimic binds to a peptide-binding groove of human leukocyte antigen (HLA) molecules to the same extent as the naturally occurring post-translationally modified peptide.

Preferably said synthetic peptide mimic is also recognized by T cells to the same degree as the post-translationally modified naturally occurring peptide.

According to an embodiment, said peptide mimic has a crystal structure determined for example by X-ray diffraction crystallography, which structure is substantially identical to a crystal structure of the naturally occurring peptide determined using the same method. Preferably said peptide mimic also binds to a peptide-binding groove of human leukocyte antigen (HLA) molecules to the same extent as the naturally occurring post-translationally modified peptide, and more preferably it is also recognized by T cells to the same degree as the post-translationally modified naturally occurring peptide.

The crystal structure of a molecule can be determined using methods and equipment available to persons skilled in the art, most commonly X-ray diffraction crystallography. A skilled person is aware of the methods and devices available for performing X-ray diffraction crystallography as the method has been practiced for several decades. For example, already the double helix structure of DNA discovered by James Watson and Francis Crick was revealed by X-ray crystallography. Similarly, the molecular binding can be studied and quantified using binding assays and associated equipment. A competitive binding assay typically measures the binding of a labelled ligand to a target protein in the presence of a second, competing but unlabelled ligand. Such assay can be used to assess qualitative binding information as well as relative affinities of two or more molecules for one target.

In the above, it is preferably an amino acid which binds to a pocket in the peptide binding groove of the HLA molecule that is substituted, for example a citrulline that is substituted with glutamine. Glutamine is herein referred to either by its full name, by a three-letter code (gln) or a one letter code (Q).

According to an embodiment of the first aspect of the invention, the synthetic peptide is a mimic of a peptide chosen from citrullinated fibrinogen, citrullinated vimentin, citrullinated collagen type II, citrullinated Cartilage Intermediate Layer Protein (CILP), citrullinated tenascin C, and citrullinated alpha-enolase.

According to a specific embodiment of the first aspect of the invention, the peptide is fibrinogen, and a citrulline in position 74 is substituted by a glutamine. The relevant sequence of the fibrinogen beta chain (amino acids 69-81) is shown as SEQ ID NO. 1 and a first mimic is illustrated by SEQ ID NO. 2.

An alternative is shown as SEQ ID NO. 3, where a tyrosine in position 71 is substituted by a phenylalanine.

According to an alternative embodiment of the first aspect of the invention, the peptide is fibrinogen, and in addition to the substitution of a citrulline in position 74 by a glutamine, a tyrosine in position 71 is substituted by a phenylalanine. This is illustrated by SEQ ID NO. 4.

According to another embodiment of the first aspect of the invention, the peptide is vimentin, and the relevant portion, a T cell epitope of the vimentin peptide, amino acids 66-78, is shown a SEQ ID NO. 5. Three synthetic peptide mimics were produced according to the invention:

According to one embodiment, the peptide is vimentin, and a citrulline in position 71 is substituted by a glutamine, as shown in SEQ ID NO. 6.

Alternatively, the peptide is vimentin, and a valine in position 68 is substituted by a phenylalanine, as shown in SEQ ID NO 7. According to yet another embodiment of the first aspect of the invention, the peptide is vimentin, and a citrulline in position 71 is substituted by a glutamine, and a valine in position 68 is substituted by a phenylalanine, as shown in SEQ ID NO. 8.

Tenascin-C is an oligomeric, multidomain matrix glycoprotein composed of six monomers. The size of these tenascin-C monomers varies from 180 to 250-300 kDa as a result of an alternative splicing of the fibronectin repeats at the pre-mRNA level. Tenascin C has recently been implicated as a target for antibodies in rheumatoid arthritis. Five potentially novel citrullinated tenascin C T cell epitopes have been identified by Song et al., 2021. Two epitopes are shown here, amino acids 871-885 (SEQ ID NO. 9) and amino acids 2067-2081 (SEQ ID NO. 10).

According to a specific embodiment of the first aspect of the invention, the peptide is tenascin C, and a citrulline in position 877 is substituted by a glutamine, as shown in SEQ ID NO. 11.

According to another specific embodiment of the first aspect of the invention, the peptide is tenascin C, and a citrulline in position 2073 is substituted by a glutamine, as shown SEQ ID NO. 12.

According to an embodiment of the first aspect and freely combinable with any embodiments thereof, the synthetic peptide binds to the P4 pocket (binding groove) of human leukocyte antigen (HLA) molecules with substantially the same affinity as the naturally occurring peptide.

This binding can be confirmed by methods known in the art, for example a fluorescence polarization-based competition assay, by investigating bound peptide-HLA complexes developed in a DELFIA® time-resolved fluorescence assay using europium-labelled streptavidin (PerkinElmer). For a description of the method, see Pieper et al., J Autoimmunity, 2018, incorporated herein by reference.

Additionally, the capacity of the peptide for being recognized by T cells is confirmed by functional T cell read-outs, i.e. a T cell reactive to the original peptide is also reacting to the synthetic mimic peptide. For a description of the method, see the experimental section of this patent application, and scientific literature, for example Boddul et al., J Trans Autoimm, 2021, incorporated herein by reference.

The novel synthetic peptide mimics presented above are expected to be useful in methods for the induction of tolerance in a subject, preferably in methods wherein the induction of tolerance is a step in the treatment, alleviation, or prevention of an autoimmune disease, such as but not limited to rheumatoid arthritis.

A second aspect of the present disclosure relates to a complex of a carrier and a peptide wherein said peptide is a synthetic mimic of a post-translationally modified naturally occurring peptide, wherein in said peptide mimic, compared to the naturally occurring peptide, a citrulline has been substituted with another amino acid, forming a peptide mimic, and wherein said peptide mimic binds to a peptide-binding groove of human leukocyte antigen (HLA) molecules to the same extent as the naturally occurring post-translationally modified peptide.

Preferably said synthetic peptide mimic is also recognized by T cells to the same degree as the post-translationally modified naturally occurring peptide.

The carrier is chosen from a nanoparticle, a protein, a blood cell, and a MHC class II molecule. The peptidomimetic peptide/peptide mimic can be bound to a carrier either alone or in complexes with other molecules, preferably MHC class II molecules or complexes containing MHC class II molecules. The MHC class II molecules are capable of binding peptides derived from intracellular proteins and displaying them at the cell surface, forming an MHC class II peptide complex. The structure and function of MHC class II peptide complexes has been extensively studied, see e.g. Dessen et al., 1997, the content of which is incorporated herein by reference.

In the above MHC class II-peptide complex, preferably a citrulline has been substituted with glutamine (Q).

According to an embodiment of said second aspect, said peptide mimic has a crystal structure determined by for example X-ray diffraction crystallography, which structure is substantially identical to a crystal structure of the naturally occurring peptide determined using the same method; wherein said peptide mimic also binds to a peptide-binding groove of human leukocyte antigen (HLA) molecules to the same extent as the naturally occurring post-translationally modified peptide, and recognized by T cells to the same degree as the post-translationally modified naturally occurring peptide.

The crystal structure of a molecule can be determined using methods and equipment available to persons skilled in the art, most commonly by X-ray diffraction crystallography. Similarly, the molecular binding can be studied and quantified using binding assays and associated equipment. A competitive binding assay typically measures the binding of a labelled ligand to a target protein in the presence of a second, competing but unlabelled ligand. This assay can be used to assess qualitative binding information as well as relative affinities of two or more molecules for one target.

According to an embodiment of the second aspect of the invention, the synthetic peptide is a mimic of a peptide chosen from citrullinated fibrinogen, citrullinated vimentin, citrullinated tenascin C, citrullinated collagen type II, Cartilage Intermediate Layer Protein (CILP), and citrullinated alpha-enolase.

According to a specific embodiment of the second aspect of the invention, the peptide is fibrinogen, and a citrulline in position 74 is substituted by a glutamine. The relevant sequence of the fibrinogen beta chain (amino acids 69-81) is shown as SEQ ID NO. 1 and a first mimic is illustrated by SEQ ID NO. 2.

An alternative is shown as SEQ ID NO. 3, where a tyrosine in position 71 is substituted by a phenylalanine.

According to an alternative embodiment of the second aspect of the invention, the peptide is fibrinogen, and in addition to the substitution of a citrulline in position 74 by a glutamine, a tyrosine in position 71 is substituted by a phenylalanine. This is illustrated by SEQ ID NO. 4.

According to another embodiment of the second aspect of the invention, the peptide is vimentin, and the relevant portion, a T cell epitope of the vimentin peptide, amino acids 66-78, is shown a SEQ ID NO. 5. Three synthetic peptide mimics were produced according to the invention:

According to one embodiment, the peptide is vimentin, and a citrulline in position 71 is substituted by a glutamine, as shown in SEQ ID NO. 6.

Alternatively, the peptide is vimentin, and a valine in position 68 is substituted by a phenylalanine, as shown in SEQ ID NO 7. According to yet another embodiment of the first aspect of the invention, the peptide is vimentin, and a citrulline in position 71 is substituted by a glutamine, and a valine in position 68 is substituted by a phenylalanine, as shown in SEQ ID NO. 8.

According to a specific embodiment of the second aspect of the invention, the peptide is tenascin C, and a citrulline in position 877 is substituted by a glutamine, as shown in SEQ ID NO. 11.

According to another specific embodiment of the second aspect of the invention, the peptide is tenascin C, and a citrulline in position 2073 is substituted by a glutamine, as shown SEQ ID NO. 12.

According to an embodiment of the second aspect and freely combinable with any embodiments thereof, the synthetic peptide binds to the P4 pocket (binding groove) of human leukocyte antigen (HLA) molecules with substantially the same affinity as the naturally occurring peptide.

This binding can be confirmed by methods known in the art, for example a fluorescence polarization-based competition assay, by investigating bound peptide-HLA complexes developed in a DELFIA® time-resolved fluorescence assay using europium-labelled streptavidin (PerkinElmer). For a description of the method, see Pieper et al., J Autoimmunity, 2018, incorporated herein by reference.

Additionally, the capacity of the peptide for being recognized by T cells is confirmed by functional T cell read-outs, i.e. a T cell reactive to the original peptide is also reacting to the synthetic mimic peptide. For a description of the method, see the experimental section of this patent application, and scientific literature, for example Boddul et al., 2021 (supra), incorporated herein by reference.

A third aspect of the invention relates to a method of inducing tolerance against a specific antigen in a subject, said method comprising a step of administering to said subject of a construct that comprises a carrier-peptide complex, wherein the peptide incorporated in said carrier-peptide complex is a synthetic peptide mimic as defined in the first aspect and embodiments thereof, presented above and in the attached claims.

A parallel aspect is a method of inducing tolerance against a specific antigen in a subject, said method comprising a step of administering to said subject of a construct that comprises an MHC class II-peptide complex, as defined in the second aspect and embodiments thereof, presented above and in the attached claims.

Preferably this induction of tolerance is a step in the treatment, alleviation, or prevention of an autoimmune disease. Regimens for tolerance induction, in particular the induction of self-tolerance, i.e. the ability of the immune system to recognize—and therefore not respond against—self-produced antigens, have recently been developed. Several systems for tolerance induction have been described in the scientific literature, so far mainly in murine models, which are currently translated into human disease. See for example Yang Y et al., Adv. Drug Deliv. Rev. 2021; Yang Y et al., Curr Opin Biotechnol, 2022 and Neef T et al., Cells, 2021, all incorporated herein by reference. However, for RA, several of these approaches are not feasible due to the lack of methods to produce the correct peptides when they contain one of more citrulline residues.

According to a preferred embodiment, said autoimmune disease is rheumatoid arthritis (RA) or an autoimmune condition that provides an increased risk for future onset of RA, said antigen is a peptide antigen, and the non-post-translationally modified peptide mimic binds to a peptide-binding groove of HLA-DRB1*04:01 and 04:04 and is recognized by T cell receptors from RA patients that are derived from T cells that are activated in RA patients.

According to an embodiment, said antigen is chosen from citrullinated fibrinogen, citrullinated vimentin, citrullinated collagen type II, citrullinated tenascin C, citrullinated CILP and citrullinated alpha-enolase.

A fourth aspect of the invention relates to a tolerogenic mRNA vaccine for inducing tolerance against a specific antigen in a subject, comprising modified, non-inflammatory mRNA encoding a non-post-translationally modified mimic of said antigen. Such a tolerogenic mRNA vaccine may be used to treat, alleviate, or prevent the development of an autoimmune disease, for example, but not limited to rheumatoid arthritis (RA).

A general introduction to mRNA vaccines is given in “mRNA vaccines manufacturing: challenges and bottlenecks” by Rosa et al., Vaccine 39 (2021) 2190-2200, incorporated herein by reference. As mRNA cannot code for citrullinated residues, these techniques for mRNA vaccination for tolerance are not feasible for RA with current knowledge about T cells involved in the pathogenesis of RA. Here, the present invention provides critical new knowledge that for the first time makes mRNA vaccination for tolerance in RA feasible.

According to an embodiment of the fourth aspect, said non-post-translationally modified mimic of an antigen is a non-citrullinated peptide which binds to a peptide-binding groove of human leukocyte antigen (HLA) molecules to the same extent as the naturally occurring post-translationally modified peptide.

Preferably said synthetic peptide mimic is also recognized by T cells to the same degree as the post-translationally modified naturally occurring peptide.

Preferably the vaccine is administered for treatment, alleviation, or prevention of rheumatoid arthritis.

According to an embodiment, said peptide mimic binds to a peptide-binding groove of human leukocyte antigen (HLA) molecules to the same extent as the naturally occurring post-translationally modified peptide, for example that said peptide mimic has a crystal structure determined for example by X-ray diffraction crystallography, which structure is substantially identical to a crystal structure of the naturally occurring peptide determined using the same method, and preferably said synthetic peptide mimic is also recognized by T cells to the same degree as the post-translationally modified naturally occurring peptide.

According to a further embodiment, freely combinable with the above, a citrulline has been substituted with another amino acid with maintained binding to the peptide-binding groove of human leukocyte antigen (HLA) molecules to the same extent as the naturally occurring post-translationally modified peptide. Preferably a citrulline has been substituted by glutamine (Q).

According to a preferred embodiment, said synthetic antigen binds to the P4 pocket (binding groove) of human leukocyte antigen (HLA) class II molecules.

According to a specific embodiment of the fourth aspect, the synthetic peptide is a mimic of an antigen chosen from citrullinated fibrinogen, citrullinated vimentin, citrullinated collagen type II, citrullinated tenascin C, citrullinated CILP and citrullinated alpha-enolase.

According to a specific embodiment of the fourth aspect of the invention, the peptide is fibrinogen, and a citrulline in position 74 is substituted by a glutamine. The relevant sequence of the fibrinogen beta chain (amino acids 69-81) is shown as SEQ ID NO. 1 and a first mimic is illustrated by SEQ ID NO. 2.

An alternative is shown as SEQ ID NO. 3, where a tyrosine in position 71 is substituted by a phenylalanine.

According to an alternative embodiment of the fourth aspect of the invention, the peptide is fibrinogen, and in addition to the substitution of a citrulline in position 74 by a glutamine, a tyrosine in position 71 is substituted by a phenylalanine. This is illustrated by SEQ ID NO. 4.

According to another embodiment of the fourth aspect of the invention, the peptide is vimentin, and the relevant portion, a T cell epitope of the vimentin peptide, amino acids 66-78, is shown a SEQ ID NO. 5. Three synthetic peptide mimics were produced according to the invention:

According to one embodiment, the peptide is vimentin, and a citrulline in position 71 is substituted by a glutamine, as shown in SEQ ID NO. 6.

Alternatively, the peptide is vimentin, and a valine in position 68 is substituted by a phenylalanine, as shown in SEQ ID NO 7. According to yet another embodiment of the first aspect of the invention, the peptide is vimentin, and a citrulline in position 71 is substituted by a glutamine, and a valine in position 68 is substituted by a phenylalanine, as shown in SEQ ID NO. 8.

According to a specific embodiment of the fourth aspect of the invention, the peptide is tenascin C, and a citrulline in position 877 is substituted by a glutamine, as shown in SEQ ID NO. 11.

According to another specific embodiment of the fourth aspect of the invention, the peptide is tenascin C, and a citrulline in position 2073 is substituted by a glutamine, as shown SEQ ID NO. 12.

According to an embodiment of the fourth aspect, freely combinable with all other embodiments of said fourth aspect, said modified, non-inflammatory mRNA is nanoparticle-formulated 1-methylpseudouridine-modified mRNA.

Such vaccines are useful in the treatment, alleviation and/or prevention of autoimmune diseases, for example, but not limited to rheumatoid arthritis.

The foregoing summary is illustrative only and is not intended to be in any way limiting. In addition to the illustrative aspects, embodiments and features described above, further aspects, embodiments and features will become apparent by reference to the following drawings, detailed description, and the examples.

### BRIEF DESCRIPTION OF THE DRAWINGS

The invention is now described, by way of example, with reference to the accompanying drawings, in which:

FIG. 1 shows a conceptual image of how a citrullinated peptide docks into the peptide binding groove of HLA DRB1*04:01, the most common MHC class II molecule associated with RA. Note how a citrulline docks into the P4 pocket of the MHC groove, and hence is not exposed towards the specific T cell receptor (TCR). The original image has previously been published in Malmström et al., Nat Rev Immunol 2017.

FIG. 2 is a graph showing the results of a peptide binding assay, also called a competition assay performed according to the method as outlined in Example 1. It is clear that the tested fibrinogen mimic peptides have the same capacity as the original citrullinated peptide to compete out an already bound reference peptide, here an influenza (HA) peptide.

FIG. 3 is a graph, showing activation of an artificial T cell line expressing a TCR specific for the citrullinated fibrinogen-peptide and showing from left to right the T-cell receptor (TCR)-dependent nuclear factor of activated T cells (NFAT) mediated activation T cells by optical fluorescence imaging (OFI) for FibF71Q74 (SEQ. ID. NO. 4), FibF71X74 (SEQ. ID. NO. 3), FibQ74 (SEQ. ID. NO. 2), FibX74 (SEQ. ID. NO. 1), and VimX71 (SEQ. ID. NO. 5), indicating that the mimic peptides are equally capable of triggering T cell activation as the original citrullinated peptide.

FIG. 4 is a graph, showing activation of an artificial T cell line expressing a TCR specific for the citrullinated fibrinogen-peptide and showing from left to right the T-cell receptor (TCR)-dependent programmed cell death protein 1 (PD1) expression for FibF71Q74 (SEQ. ID. NO. 4), FibF71X74 (SEQ. ID. NO. 3), FibQ74 (SEQ. ID. NO. 2), FibX74 (SEQ. ID. NO. 1), and VimX71 (SEQ. ID. NO. 5), indicating that the mimic peptides are equally capable of triggering T cell activation as the original citrullinated peptide.

FIG. 5 is a graph showing the results of a peptide binding assay also called competition assay for the citrullinated vimentin peptide compared to the non-post-translationally modified mimic. It is clear that the tested vimentin mimic peptides have the same capacity as the original citrullinated peptide to compete out an already bound reference peptide, here an influenza (HA) peptide.

FIG. 6 depicts the results of flow cytometry staining of polyclonal CD4+ T cells with HLA class II tetramers which captures antigen-specific T cells by binding to their TCRs. Here quadrant 2 (in bold) depicts T cells that are reactive both to the citrulline and glutamine tetramer of vimentin, implicating that there are T cells in this culture which cannot distinguish between the original and mimic peptides.

### DESCRIPTION

Before the present invention is described, it is to be understood that the terminology employed herein is used for the purpose of describing particular embodiments only and is not intended to be limiting, since the scope of the present invention will be limited only by the appended claims and equivalents thereof.

It must be noted that, as used in this specification and the appended claims, the singular forms “a,” “an,” and “the” include plural referents unless the context clearly dictates otherwise.

When referring to peptide sequences, the amino acids are numbered, wherein the number denotes the position of the amino acid residue in the polypeptide chain, when counted from the amino terminal. So, for example Glu 74 means that the 74th amino acid residue in the chain is a glutamine.

The term “peptide mimic” refers to a molecule such as a peptide, a modified peptide or any other molecule that biologically mimics the action or activity of some other peptide. A “peptide mimic” is sometimes also called a “peptide mimetic”.

The term “synthetic” is used to distinguish a modified, non-naturally occurring molecule, such as a peptide mimic, from naturally occurring molecules.

The expressions “post-translational modification” and “post-translationally modified” refer to the reversible or irreversible chemical changes peptides and proteins may undergo after translation. In other words, post-translational modifications are chemical modifications of a polypeptide chain that occur after DNA has been transcribed into RNA and translated into peptides and proteins. These chemical alterations range from the enzymatic cleavage of peptide bonds to the covalent additions of particular chemical groups, lipids, carbohydrates, or even entire proteins to amino acid side chains. (Uversky V. N., Posttranslational Modification, in Brenner's Encyclopaedia of Genetics (Second Edition) Elsevier Inc. 2013, Pages 425-430, incorporated herein by reference)

The expression “substantially identical” as for example in “a synthetic non-post-translationally modified peptide mimic having a three-dimensional structure substantially identical to that of the corresponding post-translationally modified naturally occurring peptide” means that said peptide mimic has a three-dimensional structure which is functionally identical, which is shown by the fact that it binds to a peptide-binding groove of human leukocyte antigen (HLA) molecules to the same extent as the corresponding naturally occurring post-translationally modified peptide.

The present disclosure frequently refers to the “peptide binding groove” of HLA class II molecules. It is known that the peptide binding grooves of both class I and class II HLA molecules are formed by a β-sheet floor consisting of eight anti-parallel β-sheets, packed against two anti-parallel α-helices forming a channel. In class I molecules (HLA-A, -B, and -C) the binding groove is divided into six pockets, A-F, which are defined by specific polymorphic amino acid residues that determine their topography and functionality. These class I HLA molecules typically bind peptides 8-11 amino acids in length. Compared to class I, the class II HLA-DRB1 molecules bind longer peptides of variable length, e.g. 12-15 amino acids. The most polymorphic HLA-DRB1 elements are the structural pockets that accommodate peptide positions 1 (P1), P4, P6, P7 and P9.

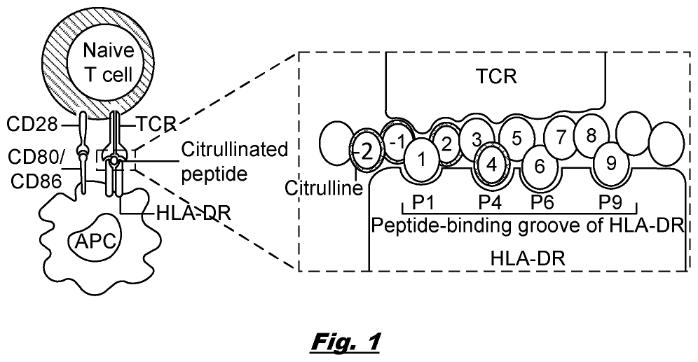

FIG. 1 shows a conceptual image of how a citrullinated peptide docks into the peptide binding groove of HLA DRB1*04:01, the most common MHC class II molecule in Caucasians associated with RA. A similar groove exists in Asian populations, then being associated with the DRB1*04:05 MHC class II variant. The original image has previously been published in Malmström et al., Nat Rev Immunol 2017.

Note how a citrulline docks into the P4 pocket of the MHC groove, and hence is not exposed towards the specific T cell receptor (TCR). A peptide mimic which is functionally identical, as defined in the present specification, examples, and claims, would thus bind to a peptide-binding groove of human leukocyte antigen (HLA) molecules to the same extent as the corresponding naturally occurring post-translationally modified peptide.

### Synthetic Peptide Mimics

Accordingly, a first aspect of the present disclosure concerns a synthetic mimic of a post-translationally modified naturally occurring peptide, wherein a citrulline has been substituted with another amino acid forming a peptide mimic, wherein said peptide mimic binds to a peptide-binding groove of human leukocyte antigen (HLA) molecules to the same extent as the naturally occurring post-translationally modified peptide.

Preferably said synthetic peptide mimic is also recognized by T cells to the same degree as the post-translationally modified naturally occurring peptide.

According to an embodiment, said peptide mimic has a crystal structure determined by for example X-ray diffraction crystallography, which structure is substantially identical to a crystal structure of the naturally occurring peptide determined using the same method. Preferably said peptide mimic also binds to a peptide-binding groove of human leukocyte antigen (HLA) molecules to the same extent as the naturally occurring post-translationally modified peptide, and most preferably said synthetic peptide mimic is also recognized by T cells to the same degree as the post-translationally modified naturally occurring peptide.

According to an embodiment of the first aspect of the invention, the synthetic peptide is a mimic of a peptide chosen from citrullinated fibrinogen, citrullinated vimentin, citrullinated collagen type II, citrullinated tenascin C, citrullinated Cartilage Intermediate layer Protein (CILP), and citrullinated alpha-enolase.

In the above, preferably a citrulline has been substituted with a glutamine (Q).

Fibrinogen in its citrullinated form is a classical candidate autoantigen in RA, and its presence has been demonstrated in the joints of RA patients by mass spectrometry analyses (Hermansson et al., 2010). It has also been suggested to form immune complexes with ACPA autoantibodies which could lead to cell activation of e.g. macrophages.

A T cell epitope from the beta chain of citrullinated fibrinogen has been identified (amino acid positions 69-81) and is widely used for detection, enumeration and phenotyping of autoreactive T cells in healthy donors and RA patients (e.g. James et al. Arthritis Rheum 2014, Gerstner et al. BMC Immunol 2020). The crystal structure of the peptide presented by HLA-DRB1*04:01 molecule has been solved (Lim et al. Sci Immunol 2021, incorporated herein by reference) and demonstrates that the citrulline is positioned in the P4 pocket as was originally predicted.

The original sequence, the fibrinogen beta chain, amino acids 69-81, is shown as SEQ. ID. NO. 1 where X denotes a citrulline, and the P1 (71) and P4 (74) positions are underlined:

(SEQ. ID. NO. 1)
GGYRAXPAKAAAT.

The present inventors synthesized three modified versions, shown below as SEQ. ID. NO. 2, 3 and 4:

(SEQ. ID. NO. 2)
GGYRAQPAKAAAT.

A glutamine in position 74.

(SEQ. ID. NO. 3)
GGFRAXPAKAAAT

A phenylalanine in position 71, and citrulline in position 74.

(SEQ. ID. NO. 4)
GGFRAQPAKAAAT

A phenylalanine in position 71, and citrulline in position 74.

Thus, according to a specific embodiment of the first aspect of the invention, the peptide is fibrinogen, and a citrulline in position 74 is substituted by a glutamine.

According to an alternative embodiment of the first aspect of the invention, the peptide is fibrinogen, and a citrulline in position 74 is substituted by a glutamine, and a tyrosine in position 71 is substituted by a phenylalanine.

Vimentin in its citrullinated form is a classical candidate autoantigen in RA, and its presence has been demonstrated in both the joints and lungs of RA patients by mass spectrometry analyses (Ytterberg et al., Ann Rheum Dis. 2015 September; 74(9):1772-7). Citrullinated vimentin has been suggested to appear on the cell surface of cells differentiating into bone resorbing osteoclasts (Harre et al., Nat Commun. 2015 Mar. 31; 6:6651).

Moreover, some ACPA autoantibodies have been shown to have a capacity to enhance both osteoclast differentiation and boost their bone resorbing capacity (Steen et al., Arthritis Rheumatol. 2019 February; 71(2):196-209; Krishnamurthy A et al., Citrullination Controls Dendritic Cell Transdifferentiation into Osteoclasts, in J Immunol. 2019 Jun. 1; 202(11):3143-3150; and Krishnamurthy A et al., Identification of a novel chemokine-dependent molecular mechanism underlying rheumatoid arthritis-associated autoantibody-mediated bone loss, Ann Rheum Dis. 2016 April; 75(4):721-9. doi: 10.1136,

A T cell epitope from citrullinated vimentin has been identified (amino acid position 66-78) and widely used for the detection, enumeration, and phenotyping of autoreactive T cells in healthy donors and RA patients (see e.g. Snir et al., Arthritis Rheum 2011, James et al., Arthritis Rheum 2014, and Gerstner et al., BMC Immunol 2020). The crystal structure of the peptide presented by HLA-DRB1*04:01 molecule has been solved (Scally et al., J Exp Med 2013) and demonstrates that the citrulline is positioned in the P4 pocket as was originally predicted.

Consequently, the inventors investigated also vimentin, and the original sequence, vimentin amino acids 66-78, is shown as SEQ. ID. NO. 5, where X denotes citrulline and the P1 and P4 positions are underlined:

(SEQ. ID. NO. 5)
SAVRLXSSVPGVR

Three modified versions were produced, shown as SEQ. ID. NO. 6, 7 and 8:

(SEQ. ID. NO. 6)
SAVRLQSSVPGVR

A glutamine in position 71.

(SEQ. ID. NO. 7)
SAFRLXSSVPGVR

A phenylalanine in position 68, and a citrulline in position 71.

(SEQ. ID. NO. 8)
SAFRLQSSVPGVR

A phenylalanine in position 68, and a citrulline in position 71.

Thus, according to another embodiment of the first aspect of the invention, the peptide is vimentin, and a citrulline in position 71 is substituted by glutamine.

According to an alternative embodiment, the peptide is vimentin, and a citrulline in position 71 is substituted by a glutamine, and a valine in position 68 is substituted by a phenylalanine.

Tenascin C in its citrullinated form is a candidate autoantigen in RA, and its presence has been demonstrated in the joints of RA patients by mass spectrometry analyses (Tutturen et al., 2014). It has also been suggested to form immune complexes with ACPA autoantibodies which could lead to cell activation of e.g. macrophages.

Several T cell epitopes from citrullinated Tenascin C has been identified and widely used for detection, enumeration, and phenotyping of autoreactive T cells in healthy donors and RA patients (e.g. Song et al., JCI Insights 2021 and Sharma et al., Sci. Rep 2021). For two of the peptides, the citrulline has been modelled to be positioned in the P4 pocket (Song et al., JCI Insights 2021)

The original sequences, Tenascin C, amino acids 871-885 and amino acids 2067-2081, are shown as SEQ. ID. NO. 9 and SEQ. ID. NO. 10 where X denotes a citrulline, and the P1 (71) and P4 (74) positions are underlined:

(SEQ. ID. NO. 9)
VSLISRXGDMSSNPA

A citrulline in position 877.

(SEQ. ID. NO. 10)
QGQYELXVDLRDHGE

A citrulline in position 2073.

Two modified versions were synthesized, shown as SEQ. ID. NO. 11 and 12:

(SEQ. ID. NO. 11)
VSLISRQGDMSSNPA

Now with a glutamine in position 877.

(SEQ. ID. NO. 12)
QGQYELQVDLRDHGE

Now with a glutamine in position 2073.

According to an embodiment of the first aspect and freely combinable with any embodiments thereof, a synthetic peptide according to the invention binds to the P4 pocket (binding groove) of human leukocyte antigen (HLA) molecules with substantially the same affinity as the corresponding naturally occurring peptide.

This binding can be confirmed by methods known in the art, for example a fluorescence polarization-based competition assay, by investigating bound peptide-HLA complexes developed in a DELFIA® time-resolved fluorescence assay using europium-labelled streptavidin (PerkinElmer). For a description of the method, see Pieper et al., J Autoimmunity, 2018, incorporated herein by reference.

Additionally, the capacity of the peptide for being recognized by T cells is confirmed by functional T cell read-outs, i.e. a T cell reactive to the original peptide is also reacting to the synthetic mimic peptide. For a description of the method, see the experimental section of this patent application, and scientific literature, for example Boddul et al., 2021 (supra), incorporated herein by reference.

The above defined peptide mimics are useful in methods for the induction of tolerance in a subject, preferably methods in which the induction of tolerance is a step in the treatment, alleviation, or prevention of an autoimmune disease, for example, but not limited to, RA.

### Peptide+Carrier Complex

A second aspect of the present disclosure relates to a complex of a carrier and a peptide wherein said peptide is a synthetic mimic of a post-translationally modified naturally occurring peptide, wherein in said peptide mimic, compared to the corresponding naturally occurring peptide, a citrulline has been substituted with another amino acid, forming said peptide mimic, and wherein said peptide mimic can bind to a peptide-binding groove of human leukocyte antigen (HLA) molecules to the same extent as the naturally occurring post-translationally modified peptide.

According to an embodiment of the second aspect and freely combinable with any embodiments thereof, the synthetic peptide binds to the P4 pocket (binding groove) of human leukocyte antigen (HLA) molecules with substantially the same affinity as the naturally occurring peptide.

This binding can be confirmed by methods known in the art, for example a fluorescence polarization-based competition assay, by investigating bound peptide-HLA complexes developed in a DELFIA® time-resolved fluorescence assay using europium-labelled streptavidin (PerkinElmer). For a description of the method, see Pieper et al., J Autoimmunity, 2018, incorporated herein by reference.

Additionally, the capacity of the peptide for being recognized by T cells is confirmed by functional T cell read-outs, i.e. a T cell reactive to the original peptide is also reacting to the synthetic mimic peptide. For a description of the method, see the experimental section of this patent application, and scientific literature, for example Boddul et al., 2021 (supra), incorporated herein by reference.

Preferably said synthetic peptide mimic is also recognized by T cells to the same degree as the post-translationally modified naturally occurring peptide.

According to an embodiment of said second aspect, said carrier is chosen from a nanoparticle, a protein, a blood cell, and an MHC class II molecule. Examples of nanoparticles include, but are not limited to, iron oxide nanoparticles, latex nanoparticles, gold nanoparticles, silica nanoparticles, and carbon nanotubes.

The carrier can be constructed both to bind only the peptidomimetic or to bind to molecular constructs containing the peptide, including for example complexes that contain peptides binding to MHC class II molecules. When MHC class II peptide complexes are included they should be able to bind peptides derived from intracellular proteins and displaying them at the cell surface, forming an MHC class II peptide complex. The structure and function of MHC class II peptide complexes has been extensively studied, see e.g. Dessen et al., 1997.

According to an embodiment of said second aspect, said peptide mimic has a crystal structure determined for example by X-ray diffraction crystallography, which structure is substantially identical to a crystal structure of the naturally occurring peptide determined using the same method; and said peptide mimic binds to a peptide-binding groove of human leukocyte antigen (HLA) molecules to the same extent as the naturally occurring post-translationally modified peptide; and said synthetic peptide mimic is also recognized by T cells to the same degree as the post-translationally modified naturally occurring peptide.

For example the crystal structure of a citrullinated fibrinogen attached to/presented by a HLA class II molecule has been published, see Lim et al., Sci Immunol 2021, supra.

In the above MHC class II-peptide complex, preferably a citrulline has been substituted with glutamine (Q).

According to an embodiment of the second aspect of the invention, the synthetic peptide is a mimic of a peptide chosen from citrullinated fibrinogen, citrullinated vimentin, citrullinated collagen type II, citrullinated tenascin C, citrullinated CILP, and citrullinated alpha-enolase.

According to a specific embodiment of the second aspect of the invention wherein the peptide is fibrinogen, and a citrulline in position 74 is substituted by a glutamine.

According to an alternative embodiment of the second aspect of the invention the peptide is fibrinogen, and a citrulline in position 74 is substituted by a glutamine, and a tyrosine in position 71 is substituted by a phenylalanine.

According to another embodiment of the second aspect of the invention the peptide is vimentin, and a citrulline in position 71 is substituted by a glutamine.

Alternatively, the peptide is vimentin, a citrulline in position 71 is substituted by a glutamine, and a valine in position 68 is substituted by a phenylalanine.

According to another embodiment of the second aspect of the invention the peptide is tenascin C, and a citrulline in position 877 is substituted by a glutamine.

According to another embodiment of the second aspect of the invention the peptide is tenascin C, and a citrulline in position 2073 is substituted by a glutamine.

The complex defined herein has utility in methods for the induction of tolerance in a subject, preferably methods wherein the induction of tolerance is a step in the treatment, alleviation, or prevention of an autoimmune disease, such as but not limited to RA.

### Tolerance Induction

A third aspect of the invention relates to a method of inducing tolerance against a specific antigen in a subject, said method comprising a step of administering to said subject of a construct that comprises a carrier-peptide complex as disclosed above, wherein the peptide is a synthetic peptide mimic as defined in the first aspect and embodiments thereof, presented above and in the attached claims.

A parallel aspect is a method of inducing tolerance against a specific antigen in a subject, said method comprising a step of administering to said subject of a construct that comprises at least one MHC class II-peptide complex, as defined in the second aspect and embodiments thereof, presented above and in the attached claims.

Preferably this induction of tolerance is a step in the treatment, alleviation or prevention of an autoimmune disease.

According to a preferred embodiment, said autoimmune disease is rheumatoid arthritis (RA), said antigen is a peptide antigen, and the non-post-translationally modified peptide mimic binds to a peptide-binding groove of HLA-DR0 401 and 0404 and is recognized by T cell receptors from RA patients that are derived from T cells that are activated in RA patients.

According to an embodiment, said antigen is chosen from citrullinated fibrinogen, citrullinated vimentin, citrullinated collagen type II, citrullinated tenascin C, citrullinated CILP and citrullinated alpha-enolase.

### Tolerogenic Vaccines

A fourth aspect of the invention relates to a tolerogenic mRNA vaccine for inducing tolerance against a specific antigen in a subject, comprising modified, non-inflammatory mRNA encoding a non-post-translationally modified mimic of said antigen.

According to an embodiment of the fourth aspect, said non-post-translationally modified mimic of an antigen is a non-citrullinated peptide which can bind to a peptide-binding groove of human leukocyte antigen (HLA) molecules to the same extent as the naturally occurring post-translationally modified peptide.

Preferably said synthetic peptide mimic is also recognized by T cells to the same degree as the post-translationally modified naturally occurring peptide.

According to an embodiment, said peptide mimic has a crystal structure determined by for example X-ray diffraction crystallography, which structure is substantially identical to a crystal structure of the naturally occurring peptide determined using the same method. Preferably, said peptide mimic also binds to a peptide-binding groove of human leukocyte antigen (HLA) molecules to the same extent as the naturally occurring post-translationally modified peptide; and preferably said synthetic peptide mimic is also recognized by T cells to the same degree as the post-translationally modified naturally occurring peptide. An example of how the crystal structure of a peptide-HLA molecule can be determined is given in Lim et al., Sci Immunol 2021, incorporated herein by reference.

According to a further embodiment, freely combinable with the above, a citrulline has been substituted with another amino acid with maintained binding to the peptide-binding groove of human leukocyte antigen (HLA) molecules to the same extent as the naturally occurring post-translationally modified peptide.

According to a preferred embodiment, said synthetic antigen binds to the P4 pocket (binding groove) of human leukocyte antigen (HLA) molecules.

According to an embodiment of the fourth aspect, a citrulline in the synthetic antigen has been substituted by another amino acid with maintained binding to the peptide-binding groove of human leukocyte antigen (HLA) molecules to the same extent as the naturally occurring post-translationally modified peptide. Preferably a citrulline has been substituted by glutamine (Q).

According to a specific embodiment of the fourth aspect, the synthetic peptide is a mimic of an antigen chosen from citrullinated fibrinogen, citrullinated vimentin, citrullinated collagen type II, citrullinated tenascin C, CILP and citrullinated enolase.

According to an embodiment of the fourth aspect, freely combinable with all other embodiments of said fourth aspect, said modified, non-inflammatory mRNA is nanoparticle-formulated 1-methylpseudouridine-modified mRNA.

The inventions disclosed herein make it possible to design novel mRNA vaccines. For example, mRNA vaccines can be designed so that mRNA when administered to a subject in need thereof is able to mediate production of the peptidomimetic peptide/peptide mimic in a non-immunogenic and tolerogenic fashion in subjects with an existing immune response. The response can be measured using known methods, for example T or B cell assays or both, against the citrullinated peptides for which the peptide mimic is a substitution. Similar to that what has been shown in other experimental contexts such as in the mIG induced EAE model (Krienke C. et al., Science 2021, incorporated herein by reference), such mRNA vaccines coding for any of the peptide mimics described above may by “bystander suppression” exert a suppressing function also for other specific immunities targeting the same organ and/or working in the same disease as the immunity against the citrullinated peptides which formed the basis for the generation of the peptidomimetic peptide.

The present inventors have checked the sequences of the modified peptides presented herein to determine if they occur in other human proteins, and so far, the investigation indicates that they do not. The present inventors have thus made available previously unknown, non-naturally occurring, non-post-translational peptides that mimic the function and structure of citrullinated peptides that play a role in the pathogenesis of RA. This opens up new possibilities for the development of new methods of immunotherapy for the treatment and prevention of autoimmune diseases, in particular RA.

In the following examples, the inventors present experimental evidence supporting the aspects and embodiments of the present invention.

### EXAMPLES

### Example 1. Non-Post-Translationally Modified Fibrinogen Mimic

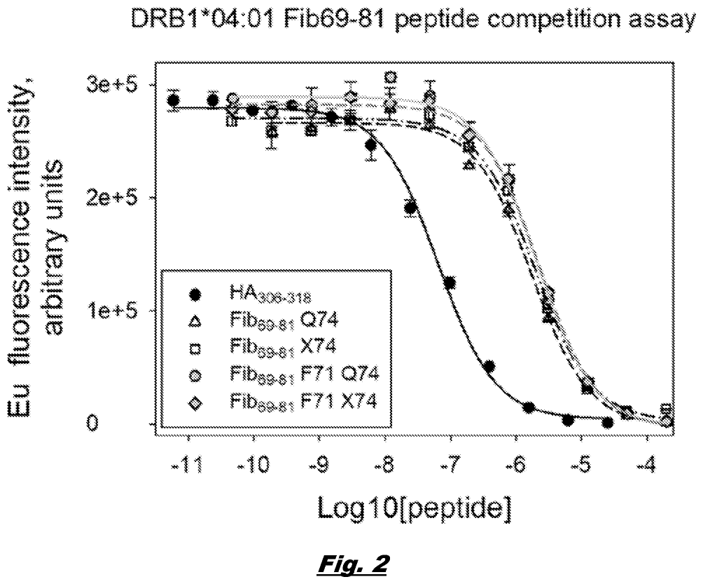

The present inventors performed binding assays to establish that the modified peptides indeed can both bind the relevant HLA molecule and present peptides to the T cell receptor (TCR) in a functional manner. The HLA binding was demonstrated in competition assays and showed an equal capacity of the mimic peptides as for the original citrullinated peptide to compete out an already bound reference peptide (in this case an influenza (HA) peptide). See FIG. 2. The results can also be presented as curves or as Kd values, demonstrating that the amino acid exchanges at positions P1 and P4 do not alter their possibility of being presented to T cells.

The inventors have previously produced data of TCR re-expression into a TCR-deficient T cell line (58−/−) for studies of antigen specificity and T cell activation (Boddul et al., 2021, supra), and have now re-expressed cit-fib-specific TCR into the same system.

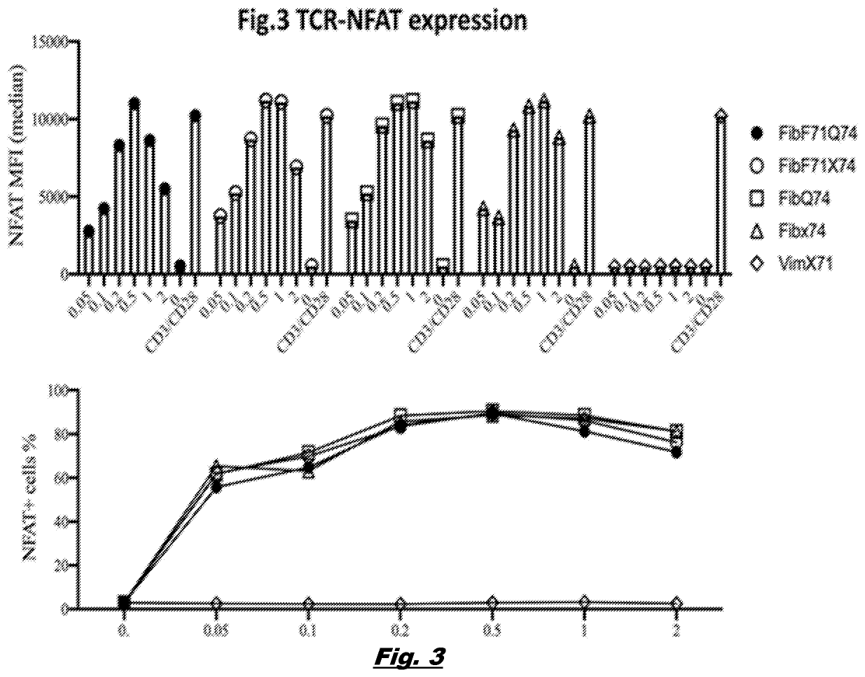

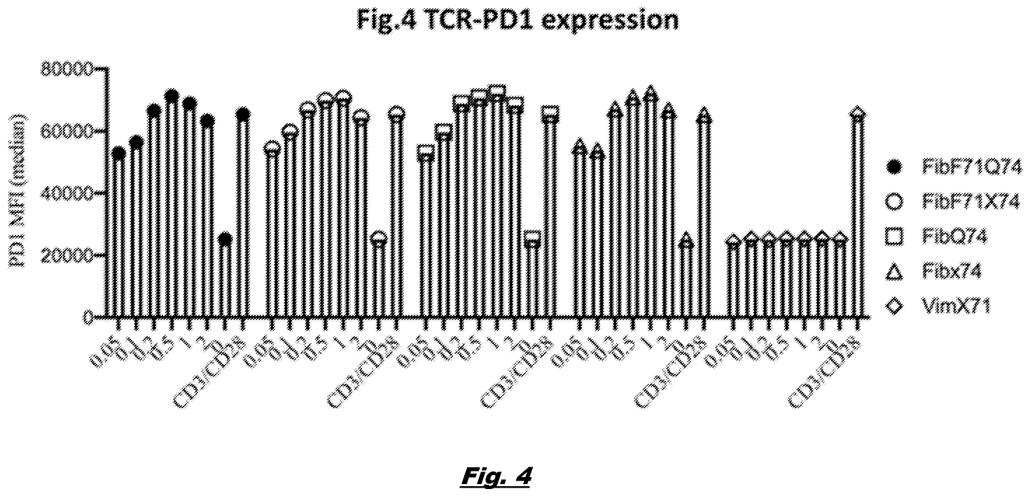

Now, the inventors demonstrated that a TCR specific for the citrullinated fibrinogen peptide cannot distinguish between the cognate peptide and the mimic peptides as the artificial T cell line responds equally with both nuclear factor of activated cells (NFAT) signalling. See FIG. 3. Similarly, the peptide mimics were compared with regard to programmed death-1 (PD-1) expression. See FIG. 4.

### Materials and Methods

58−/− cell line expressing cit-fib specific TCRs in ametrine expressing vector, along with co-expression of human CD4 and GFP as a reporter for NFAT expression.

HLA-DRB1*04:01 monomeric protein (500 μg/mL) was incubated for 72 h at 37° C. with different versions of fib69-81 peptide (test) and VimX71 peptide (control) in sodium phosphate buffer (1×) containing n-octyl β-D-Glucopyranoside (Sigma-Aldrich, USA) and Pefabloc SC (Sigma-Aldrich, USA), and then stored at 4° C. until used. Loaded HLA monomers were subsequently coated (0.03-2 μg/well) onto 48-well plates in 100 μl PBS for 4 h at 37° C. The HLA/peptide solution was subsequently flicked of the plate and specific T cells (5×104) and anti-CD28 (1 μg/well) were added to the monomer-coated wells and incubated for 48 h at 37° C. before collecting cells.

As positive controls, anti-mouse anti-CD3 (BioLegend #100314), anti-CD28 (BioLegend #101112) were used.

PD1 expression on 58−/− cells was assessed using anti-mouse PD-1 PE-Cy7 antibody, while NFAT activation was studied using assessment of GFP expression following cell stimulation. Human CD4+ametrine+ viable singlets were used as population to look for expression of NFAT and PD1.

A competitive binding assay, see for example, was used to demonstrate the capacity of the mimic peptide to bind HLA-DRB1*04:01 compared to the original citrullinated peptide. The instrument was a PerkinElmer 1420 Multilabel Counter VICTOR3™ V using the settings shown in the table below:

| Name of the plate type | NUNC 384 |
|---|---|
| Number of wells in the plate | 16 × 24 |

| | | |
|---|---|---|
| Height of the plate | 14.4 | mm |

| Offset of the wells | 9.000 mm, 12.000 mm |
|---|---|
| Distance between wells | 4.500 mm, 4.500 mm |
| Number of repeats | 1 |

| | | |
|---|---|---|
| Delay between repeats | 0 | s |
| Measurement height | 8.00 | mm |

| | |
|---|---|
| Name of the label | Europium |
| Label technology | Time resolved fluorometry |
| Emission filter name | D615 |
| Emission filter slot | A1 |
| Emission aperture | Normal |
| Excitation filter | D340 |

| | | |
|---|---|---|
| Delay | 400 | μs |
| Window time | 400 | μs |
| Cycle time | 1000 | μs |
| Second window delay time | 0 | μs |
| Second window time | 0 | μs |

| Light integrator capacitors | 1 |
|---|---|
| Light integrator ref. level | 141 |
| Flash energy area | Low |
| Flash energy level | 31 |
| Flash absorbance measurement | No |
| Beam | Normal |

The lines in FIG. 2 represent fitting of experimental data to the one site competition model using SigmaPlot software v.13 (Systat Software, Inc., San Jose, California, USA).

Increasing concentrations of the peptides were incubated in 384-well polypropylene plate in the presence of 30 nM HLA-DRB1*04:01 and 5 nM biotin-labelled HA306-318 peptide overnight at 37° C. in a humidified incubator. Reaction mixture was transferred to a polystyrene plate coated with anti-HLA-DR mAb L243 and incubated overnight at +4 C. Bound peptide-HLA complexes were developed in DELFIA® time-resolved fluorescence assay using europium-labelled streptavidin (PerkinElmer)

The results show that the T cell line cannot distinguish peptide presentation of the citrulline-containing peptide from the glutamine-containing (artificial) peptide when presented on HLA-DRB1*04:01. The cells responded equally well by NFAT signalling and PD1 upregulation.

In order to further increase the stability of the peptide-HLA complex the inventors also replaced the amino acid in the P1 pocket (which also will not be exposed to the TCR) and also these peptides could trigger the T cells, while irrelevant peptides presented by the same HLA-DRB1*04:01 molecule does not activate the cells.

The results show that the T cell line cannot distinguish peptide presentation of the citrulline-containing peptide from the glutamine-containing and P1 optimised (artificial) peptide when presented on HLA-DRB1*04:01. The cells responded equally well by NFAT signalling and PD1 upregulation.

### Example 2. Non-Post-Translationally Modified Vimentin Mimic

A T cell epitope from citrullinated vimentin has previously been identified (amino acid position 66-78) and widely used for detection, enumeration, and phenotyping of autoreactive T cells in healthy donors and RA patients (e.g. Snir et al., Arthritis Rheum 2011, James et al., Arthritis Rheum 2014, and Gerstner et al., BMC Immunol 2020). The crystal structure of the peptide presented by HLA-DRB1*04:01 molecule has been solved (Scally et al., J Exp Med 2013) and demonstrates that the citrulline is positioned in the P4 pocket as was originally predicted.

### Materials and Methods

Peptide competition was performed in the same manner as for the fibrinogen peptides, see the materials and methods in Example 1, mutatis mutandis.

Primary cells from RA patients have been in vitro cultivated with the original citrullinated vimentin peptide for 14d. Cell medium supplemented with human serum and 50 U of recombinant IL-2 from day 5. 37° C. incubator with 5% CO2.

Upon harvest, the cells have been centrifuged and the pellet resuspended in PBS and stained with HLA class II tetramers, which are reagents that can only interact with T cells carrying a TCR capable of interacting with the peptide-HLA complex. Here two sets of tetramers were used for staining (in different color).

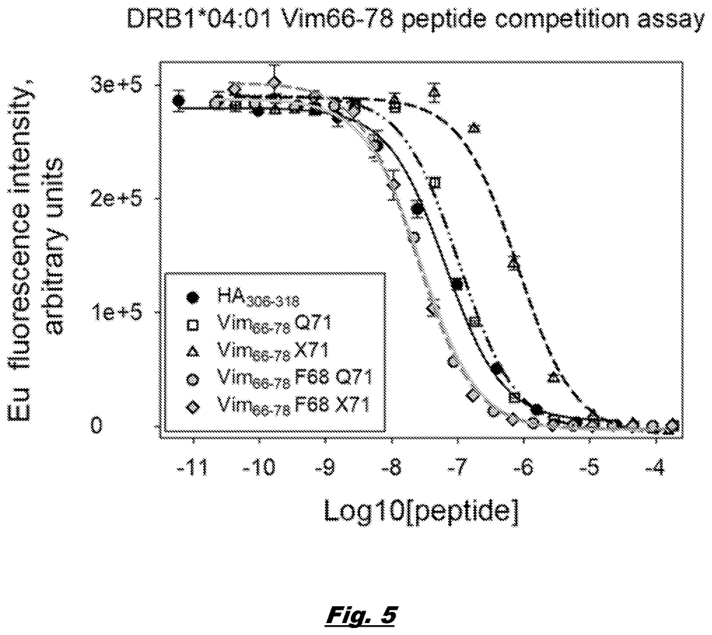

The inventors performed binding assays to establish that the modified peptides indeed can bind and thereby present peptides to the TCR, these are competition assays and show the capacity of a tested peptide to compete out an already bound peptide (in this case an influenza (HA) peptide). The results can be presented as curves or as Kd values, both are depicted in the attached figures and demonstrates that a single amino acid exchange at positions P4 reduces the Kd value while the double exchange has better (lower) Kd values. See FIG. 5. Nota bene these numbers only reflect the capacity of a peptide to bind the HLA and not the interaction with TCR.

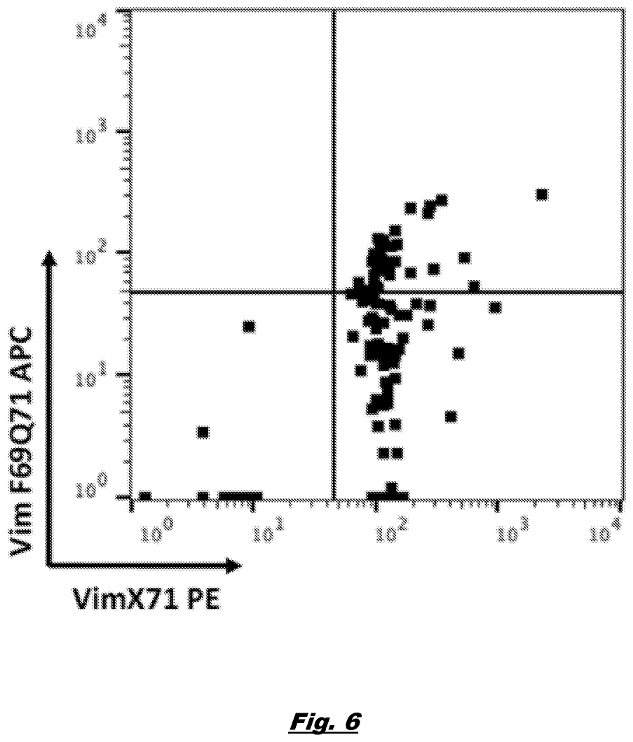

Further, short term T cell lines from primary cells of RA patients were generated and the inventors demonstrated that many of the cit-specific T cells identified by peptide-HLA tetramers also bound the tetramer loaded with the glutamine-version of the peptide. In FIG. 6, the cit-peptide reactive T cells are found on the x-axis and the glutamine reactive T cells on the y-axis. The results clearly show that there are T cells which cannot distinguish between the original and mimic peptides

Additionally, the inventors have sequenced the TCR from these cells and generated T cell lines and commenced studies as already described for the fibrinogen peptide.

In summary, the new insights presented herein can be used to develop methods for generating antigen-specific tolerance, wherein the inventive peptides are administered either alone, complexed with relevant MHC class II molecules, attached to other molecular or cellular complexes and in some cases attached to a suitable carrier, such as a nanoparticle, a protein, or a blood cell, to mention some examples. Knowledge of these peptides can also be used in the design of tolerizing mRNA vaccines. Such vaccines can, for example, be produced in similar ways as described for mRNA vaccines coding for MOG peptides for tolerance therapy for experimental allergic encephalomyelitis (EAE) (Krienke C. et al., Science 2021, incorporated herein by reference).

This principle of defining and producing alternative non-post translationally modified peptide mimics will significantly improve the production of tolerogenic molecular constructs and appropriate mRNA molecules and thus provide a major breakthrough for the development of tolerizing therapies for autoimmune diseases and in particular RA.

The concept of making alternative and not naturally occurring non-post-translationally modified peptide mimics instead of naturally occurring post-translationally modified peptides and using these for therapeutic purposes potentially opens up a new therapeutic principle, where the design of therapeutic peptides is greatly improved for therapy or prevention of immune-mediated diseases driven by immunity against post-translationally modified proteins and peptides.

Additionally, so called “tolerogenic particles’ designed for use as bio pharmaceuticals and administered to patients can now be produced in ways that make these pharmaceuticals more stable, easier to produce including producing them with higher quality, as well as new, straight-forward ways to assure this this increased quality. The contribution of the present inventors represents major and unexpected advances related to production of tolerogenic medicines for autoimmune diseases, such as, but not limited to, RA.

Without further elaboration, it is believed that a person skilled in the art can, using the present description, including the examples, utilize the present invention to its fullest extent. Also, although the invention has been described herein with regard to its preferred embodiments, which constitute the best mode presently known to the inventors, it should be understood that various changes and modifications as would be obvious to one having the ordinary skill in this art may be made without departing from the scope of the invention which is set forth in the claims appended hereto.

Thus, while various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

### SEQUENCE LISTING

(SEQ. ID. NO. 1)
GGYRAXPAKAAAT
(SEQ. ID. NO. 2)
GGYRAQPAKAAAT
(SEQ. ID. NO. 3)
GGFRAXPAKAAAT
(SEQ. ID. NO. 4)
GGFRAQPAKAAAT
(SEQ. ID. NO. 5)
SAVRLXSSVPGVR
(SEQ. ID. NO. 6)
SAVRLQSSVPGVR
(SEQ. ID. NO. 7)
SAFRLXSSVPGVR
(SEQ. ID. NO. 8)
SAFRLQSSVPGVR
(SEQ. ID. NO. 9)
VSLISRXGDMSSNPA
(SEQ. ID. NO. 10)
QGQYELXVDLRDHGE
(SEQ. ID. NO. 11)
VSLISRQGDMSSNPA
(SEQ. ID. NO. 12)
QGQYELQVDLRDHGE
(Amino acid sequences, one-letter code)

### REFERENCES

Boddul S V, Sharma R K, Dubnovitsky A, Raposo B, Gerstner C, Shen Y, Iyer V S, Kasza Z, Kwok W W, Winkler A, Klareskog L, Malmström V, Bettini M and Wermeling F. In vitro and ex vivo functional characterization of human HLA-DRB1*04 restricted T cell receptors. 2021 J Trans Autoimm, March 3; 4:100087
Dessen A, Lawrence C M, Cupo S, Zaller D M, Wiley D C, X-ray crystal structure of HLA-DR4 (DRA* 0101, DRB1* 0401) complexed with a peptide from human collagen II, Immunity, 1997—Elsevier
Gerstner C, Turcinov S, Chemin K, Uchtenhagen H, Ramwadhdoebe T H, Dubnovitsky A, Tandre K, Rönnblom L, Kwok W, James E A, Catrina A I, Achour A, van Baarsen E M and Malmstrom V. Multi HLA-class II tetramer analyses of citrulline—reactive T cells and early treatment response in rheumatoid arthritis. 2020 BMC Immunol 21:27
Harauz G. and Musse A. A. et al., A tale of two citrullines-structural and functional aspects of myelin basic protein deamination in health and disease, Neurocehm Res, 2006, 32, 137-158
Harre et al., Nat Commun. 2015 Mar. 31; 6:6651. doi: 10.1038/ncomms7651.PMID: 25825024
Hermansson M, Artemenko K, Ossipova E, Eriksson H, Lengqvist J, Makrygiannakis D, Catrina A I, Nicholas A P, Klareskog L, Savitski M, Zubarev R A, Jakobsson P J. MS analysis of rheumatoid arthritic synovial tissue identifies specific citrullination sites on fibrinogen. Proteomics Clin Appl 2010 5:511-8
James E, Rieck M, Pieper J, Gebe J A, Yue B B, Tatum M, Peda M, Sandin C, Klareskog L, Malmstrom V and Buckner J H. Citrulline specific CD4+ T cells exhibit a Th1 memory phenotype in R A subjects and their ex vivo frequency is influenced by both disease duration and biologic therapy. 2014 Arthritis Rheum 66:1712-22
Krienke C. et al., A noninflammatory mRNA vaccine for treatment of experimental autoimmune encephalomyelitis, Science 371, 145-153 (2021)
Krishnamurthy A, Joshua V, Haj Hensvold A, Jin T, Sun M, Vivar N, Ytterberg A J, Engström M, Fernandes-Cerqueira C, Amara K, Magnusson M, Wigerblad G, Kato J, Jiménez-Andrade J M, Tyson K, Rapecki S, Lundberg K, Catrina S B, Jakobsson P J, Svensson C, Malmström V, Klareskog L, Wähämaa H, Catrina A I, Identification of a novel chemokine-dependent molecular mechanism underlying rheumatoid arthritis-associated autoantibody-mediated bone loss, in Ann Rheum Dis. 2016 April; 75(4):721-9. doi: 10.1136
Krishnamurthy A, Ytterberg A J, Sun M, Sakuraba K, Steen J, Joshua V, Tarasova N K, Malmström V, Wähämaa H, Réthi B, Catrina A I. Citrullination Controls Dendritic Cell Transdifferentiation into Osteoclasts, in J Immunol. 2019 Jun. 1; 202(11):3143-3150.
Lim J J, Jones C M, Loh T J, Ting Y T, Zarei P, Loh K L, Felix N J, Suri A, Mckinnon M, Stevenaert F, Sharma R K, Klareskog L, Malmström V, Baker D G, Purcell A W, Reid H H, La Gruta N L and Rossjohn J. The shared susceptibility epitope of HLA_DR4 binds citrullinated self-antigens and the TCR. 2021 Sci Immunol. in press
Malmstrom V, Catrina A I, and Klareskog L. The immunopathogenesis of seropositive rheumatoid arthritis: from triggering to targeting. 2017 Nat Rev Immunol 17:60-75
Neef T, Ifergan I, Beddow S, Penaloza-MacMaster P, Haskins K, Shea L D, Podojil J R, Miller S D. Tolerance Induced by Antigen-Loaded PLG Nanoparticles Affects the Phenotype and Trafficking of Transgenic CD4+ and CD8+ T Cells, in Cells. 2021 Dec. 7; 10(12):3445
Nel H J, Malmström V, Wraith D C and Thomas R. Autoantigens in rheumatoid arthritis and the potential for antigen-specific tolerizing immunotherapy. Lancet Rheumatology 2020 2:e712-23
Pieper J, Dubnovitsky A, James E, Gerstner C, Rieck M, Gebe J A, Achour A, Buckner J H and Malmström V. HLA-DR*0401 restricted T cells specific for the citrullinated RA autoantigen alpha-enolase are enriched in the rheumatic joint. 2018 J Autoimmunity 92:47-56.
Rosa S S, Duarte M. F. Prazeres, Ana M. Azevedo, Marco P. C. Marques, mRNA vaccines manufacturing: challenges and bottlenecks, Vaccine 39 (2021) 2190-2200.
Scally S W, Petersen J, Law S C, Dudek N L, Nel H J, Loh K L, Wijeyewickrema L C, Eckle SBG, van Heemst J, Pike R N, Mccluskey J, Toes R E, La Gruta N L, Purcell A W, Reid H H, Thomas R and Rossjohn J. A molecular basis for the association of the HLA-DRB1 locus, citrullination and rheumatoid arthritis. J Exp Med 2013 210:2569-82
Sharma R K et al., Biased TCR gene usage in citrullinated Tenascin C specific T-cells in rheumatoid arthritis, Sci Rep. 2021 Dec. 31; 11 (1): 24512
Snir O, Rieck M, Gebe J A, Yue B B, Rawlings C A, Nepom G, Malmström V and Buckner J H. Identification and functional characterization of T cells reactive to citrullinated vimentin in HLA-DRB1*0401 humanized mice and rheumatoid arthritis patients. Arthritis Rheum 2011 63:2873-2883.
Song J, Schwenzer A, Wong A, Turcinov S, Rims C. Shared recognition of citrullinated tenascin-C peptides by T and B cells in rheumatoid arthritis, JCI insight, 2021
Steen et al., Arthritis Rheumatol. 2019 February; 71(2):196-209. doi: 10.1002/art.40699.PMID: 30152202
Sun et al., Ann Rheum Dis. 2019 December; 78(12):1621-1631. doi: 10.1136/annrheumdis-2018-214967. Epub 2019 Sep. 3.PMID: 31481351.
Tutturen A E, et al. Assessing the citrullinome in rheumatoid arthritis synovial fluid with and without enrichment of citrullinated peptides. J Proteome Res. 2014; 13(6):2867-2873.
Uversky V N, Posttranslational Modification, in Brenner's Encyclopedia of Genetics (Second Edition) Elsevier Inc. 2013, Pages 425-430
Yang Y, Santamaria P., Evolution of nanomedicines for the treatment of autoimmune disease: From vehicles for drug delivery to inducers of bystander immunoregulation, in Adv Drug Deliv Rev. 2021 September; 176:113898. doi: 10.1016
Yang Y, Santamaria P., Antigen-specific nanomedicines for the treatment of autoimmune disease: target cell types, mechanisms and outcomes, in Curr Opin Biotechnol. 2022 April; 74:285-292
Ytterberg et al., Ann Rheum Dis. 2015 September; 74(9):1772-7. doi: 10.1136/annrheumdis-2013-204912. Epub 2014 May 9.PMID: 24817415.

1. A peptide mimic of a post-translationally modified naturally occurring peptide for use in a method for the induction of tolerance in a subject, wherein the induction of tolerance is a step in the treatment, alleviation, or prevention of an autoimmune disease, wherein, compared to the corresponding post-translationally modified naturally occurring peptide, a citrulline has been substituted with another amino acid forming the peptide mimic, wherein said peptide mimic binds to a peptide-binding groove of a human leukocyte antigen (HLA) molecule to the same extent as the corresponding naturally occurring post-translationally modified peptide, and wherein said peptide mimic is recognized by T cells to the same extent as the corresponding naturally occurring post-translationally modified peptide.

2. (canceled)

3. The peptide mimic according to claim 1, wherein the synthetic peptide is a mimic of a peptide chosen from citrullinated fibrinogen, citrullinated vimentin, citrullinated collagen type II, citrullinated Cartilage Intermediate Layer Protein (CILP), citrullinated tenascin C and citrullinated alpha-enolase.

4. The peptide mimic according to claim 3, wherein the peptide is fibrinogen, and a citrulline in position 74 is substituted by a glutamine.

5. The peptide mimic according to claim 3, wherein the peptide is fibrinogen, and a citrulline in position 74 is substituted by a glutamine, and a tyrosine in position 71 is substituted by a phenylalanine.

6. The peptide mimic according to claim 3, wherein the peptide is vimentin, and a citrulline in position 71 is substituted by a glutamine, optionally wherein a valine in position 68 is substituted by a phenylalanine.

7. (canceled)

8. The peptide mimic according to claim 3, wherein the peptide is tenascin C, and a citrulline in position 877 is substituted by a glutamine.

9. The peptide mimic according to claim 3, wherein the peptide is tenascin C, and a citrulline in position 2073 is substituted by a glutamine.

10. (canceled)

11. A complex of a carrier and a peptide wherein said peptide is a mimic of a corresponding post-translationally modified naturally occurring peptide, wherein compared to the naturally occurring peptide, a citrulline has been substituted with another amino acid forming a peptide mimic, wherein said peptide mimic binds to a peptide-binding groove of a human leukocyte antigen (HLA) molecule to the same extent as the corresponding naturally occurring post-translationally modified peptide, and wherein said peptide mimic is recognized by T cells to the same extent as the naturally occurring post-translationally modified peptide, wherein said carrier is chosen from a nanoparticle, a blood cell, and an MHC class II molecule.

12. (canceled)

13. The complex according to claim 11, wherein the peptide is a mimic of a peptide chosen from citrullinated fibrinogen, citrullinated vimentin, citrullinated collagen type II, citrullinated tenascin C, citrullinated Cartilage Intermediate Layer Protein (CILP), and citrullinated alpha-enolase.

14. The complex according to claim 11, wherein the peptide is fibrinogen, and a citrulline in position 74 is substituted by a glutamine as set forth in SEQ ID NO. 2.

15. The complex according to claim 11, wherein the peptide is fibrinogen, and a citrulline in position 74 is substituted by a glutamine, and a tyrosine in position 71 is substituted by a phenylalanine as set forth in SEQ ID NO. 4.

16. The complex according to claim 11, wherein the peptide is vimentin, and a citrulline in position 71 is substituted by a glutamine as set forth in SEQ ID NO. 6, optionally wherein a valine in position 68 is substituted by a phenylalanine as set forth in SEQ ID NO. 8.

17. (canceled)

18. The complex according to claim 11, wherein the peptide is tenascin C, and a citrulline in position 877 is substituted by a glutamine as set forth in SEQ ID NO 11.

19. The complex according to claim 11, wherein the peptide is tenascin C, and a citrulline in position 2073 is substituted by a glutamine as set forth in SEQ ID NO 12.

20. (canceled)

21. A method of inducing tolerance against a specific antigen in a subject, said method comprising a step of administering to said subject of a construct comprising a carrier and a peptide, wherein the peptide incorporated in said carrier-peptide complex is a peptide mimic according to claim 1.

22. The method according to claim 21, wherein said construct is a MHC class II-peptide complex.

23. (canceled)

24. The method according to claim 21, wherein the induction of tolerance is a step in the treatment, alleviation or prevention of an autoimmune disease.

25. The method according to claim 24, wherein the autoimmune disease is rheumatoid arthritis, said antigen is a peptide antigen, and wherein the non-post-translationally modified peptide mimic binds to a peptide-binding groove of HLA-DR0 401 and 0404 and is recognized by T cell receptors from RA patients that are derived from T cells that are activated in RA patients, wherein the antigen is chosen from citrullinated fibrinogen, citrullinated vimentin, citrullinated collagen type II, citrullinated tenascin C, citrullinated Cartilage Intermediate Layer Protein (CILP), and citrullinated alpha-enolase.

26. (canceled)

27. A tolerogenic mRNA vaccine for inducing tolerance against a specific antigen in a subject, comprising modified, non-inflammatory mRNA encoding a non-post-translationally modified mimic of said antigen, wherein said non-post-translationally modified mimic of said antigen is a peptide mimic binds to a peptide-binding groove of human leukocyte antigen (HLA) molecules to the same extent as the naturally occurring post-translationally modified peptide.

28. The vaccine according to claim 27, wherein in said peptide, a citrulline has been substituted by another amino acid with maintained binding to the peptide-binding groove of human leukocyte antigen (HLA) molecules to the same extent as the naturally occurring post-translationally modified peptide.

29. (canceled)

30. The vaccine according to claim 27, wherein a citrulline has been substituted by glutamine.

31. The vaccine according to claim 27, wherein the peptide is a mimic of an antigen chosen from citrullinated fibrinogen, citrullinated vimentin, citrullinated collagen type II, citrullinated tenascin C, citrullinated Cartilage Intermediate Layer Protein (CILP), and citrullinated enolase.

32. The vaccine according to claim 27, wherein said modified, non-inflammatory mRNA is nanoparticle-formulated 1-methylpseudouridine-modified mRNA

33. (canceled)

34. (canceled)
